(19) United States
(12) Patent Application Publication
(10) Pub. No.: US 2014/0363890 A1
(43) Pub. Date: Dec. 11, 2014

(54) THREE-DIMENSIONAL STRUCTURES FOR CELL OR TISSUE CULTURE

(71) Applicant: SNS Nano Fiber Technology, LLC., Hudson, OH (US)

(72) Inventors: Iksoo Chun, Princeton, NJ (US); Laura M. Frazier, Stow, OH (US); Woraphon Kataphinan, Akron, OH (US)

(21) Appl. No.: 14/298,550

(22) Filed: Jun. 6, 2014

Related U.S. Application Data
(60) Provisional application No. 61/832,074, filed on Jun. 6, 2013.

(51) Int. Cl. C12N 5/00; D01D 5/04; D01D 5/08; D01D 5/00

(52) U.S. Cl. CPC C12N 5/0068; D01D 5/00; D01D 5/0007; D01D 5/04; D01D 5/08; C12N 2535/00
USPC 435/377; 435/398; 264/6; 264/10; 264/12; 264/13

(57) ABSTRACT

Among others, the present invention provides devices for cell or tissue culture, comprising a three-dimensional structure, which further includes fibrils with beads and/or particles. The present invention also relates to novel methods for manufacturing devices for cell or tissue culture.

### CROSS-REFERENCE TO RELATED APPLICATION

This application claims priority to U.S. Patent Application No. 61/832,074, filed on Jun. 6, 2013, the contents of which are incorporated herein by reference in their entireties.

### BACKGROUND OF THE INVENTION

Two-dimensional plastic dishes have been used as a standard device for cell or tissue culture for several decades. However, such two-dimensional plastic dishes cannot mimic natural extracellular matrices environments. Compared to two-dimensional cell culture systems, the cellular activities and cell shapes observed in three-dimensional cell culture systems are more similar to the results from in vivo. Therefore, especially due to the recent developments in the life sciences such as regenerative medicine and pharmaceutical research, there is an increasing demand for cell culture devices with three-dimensional structures.

Current cell or tissue culture devices that are alleged to involve three-dimensional structures are mostly manufactured by an electrospinning method. The electrospinning method is capable of producing fibers, which can be collected as a nonwoven. However, these conventional electrospun fibrous devices have many disadvantages for use as cell or tissue culture. For example, the electrospun nonwovens usually have small pores—in a size range of microns, which are too small for cells to penetrate fully into a cell culture device. In addition, the electrospun nonwoven is so thin that such cell culture devices cannot provide a real three-dimensional environment. Furthermore, it is very difficult to handle the thin electrospun nonwoven because it is easily folded during in vito and in vivo experiments. Recently, there have been some efforts to layer multiple layers together in order to make an electrospun nonwoven thicker. However, delamination then becomes an issue, which has not been resolved by researchers thus far. These disadvantages can be overcome by the present invention.

### SUMMARY OF THE INVENTION

The present invention in general provides a cell or tissue culture device containing a three-dimensional structure, which further comprises fibrils with beads. The beads can be either entirely or partially incorporated into the fibrils, the beads can be chemically or physically attached on/to the surface of fibrils, or the beads can be dispersed in the space between fibrils. The device provided by the present invention can be either absorbable or non-absorbable.

As used herein, the term “beads” can be interchanged with the term “particles.”

In some embodiments, the three-dimensional structure contained in a device of this invention provides a controllable and open (not sealed) cell culture system. For example, the three-dimensional structure can be fluffy and contain open large pores.

In some embodiments, the fibrils contained in a device of this invention can be the same as or different from each other, either in their chemical composition or in a physical property. Similarly, the beads contained in a device of this invention can be the same or different, either in their chemical composition or a physical property (e.g., water affinity, mechanical strength, biodegradability, molecular weight, or size).

In some embodiments, the fibrils contained in a device of this invention comprise a biocompatible material or a biodegradable material. In some other embodiments, the fibrils are biopolymers. In some embodiments, the fibrils can be synthetic or natural polymers, such as collagen, gelation, chitosan, and zein.

In some other embodiments, the fibrils are oligomers, prepolymers, or monomers. In some embodiments, the fibrils are polymeric materials, such as polystyrene, polyolefin, polysaccharides, collagen, gelatin, zein, polyvinylpyrrolidone, hydroxypropyl methyl cellulose, polyethylene oxide, polyethylenimine polyvinyl alcohol, polyamides, or polyurethanes. In some embodiments, the fibrils can be aliphatic polyesters. Examples of suitable aliphatic polyesters include polycarprolactone (e.g., poly(ε-caprolactone)), poly(lactate), poly(glycolate), poly(dioxanone), polyhydroxyallanoates and their copolymers. These aliphatic polyesters are among the few synthetic polymers approved by the US Food and Drug Administration (FDA) for certain human clinical applications such as surgical sutures and some implantable devices. In one embodiment, the fibrils contained in a device of the present invention are fabricated or manufactured from an aliphatic polyester suitable for in vivo human applications.

In some embodiments, the fibrils are nanofibers. In some other embodiments, the fibrils are surface-treated by physical, chemical, biological treatment such as plasma treatment or, with a biocompatible material.

In some embodiments, the beads in a device of this invention can be (1) cells' beds; (2) storage areas or space for bioactive components such as growth factors, differentiation factors, pharmaceutical small molecules, biological large molecules, or cell adhesion molecules; (3) supports to retain a 3-dimensional structure of the device when the device is saturated; and/or (4) pore openers when they are swollen. For example, the beads can be active in order to enhance cell spreading, cell attachment, cell growth, and the differentiation of cells.

The beads contained in a device of the present invention can be non-cytotoxic. In some embodiments, the beads comprise organic, inorganic, synthetic, or natural materials. For example, the beads can be made, either entirely or partially, of such material as glass, bioactive molecule, growth factor, differentiation factor, cell adhesion molecule(s), pharmaceutical small molecule(s), biological large molecule(s), or absorbent particle(s). Alternatively, the beads may be incorporated with/comprised of bioactive molecules, growth factors, differentiation factors, cell adhesion molecules or proteins, pharmaceutical small molecules or absorbent particles. For example, beaded forms of bioactive molecules, growth factors, differentiation factors, cell adhesion molecules/proteins, pharmaceutical small molecules, or absorbent particles can be incorporated during a spinning process.

In some other embodiments, the beads comprise powders or spheres of natural materials such as collagen, gelatin, chitosan, or zein. The sources of such natural materials can be human, animals, or vegetables.

Examples of bioactive molecules suitable to be included in the beads include, but are not limited to, growth factors, differentiation factors, fibrous proteins, and adhesive proteins. Examples of the growth factors include vascular endothelial growth factor (VEGF), collagen, bone morphogenic factor-β, epidermal cell growth factor (EGF), platelet derived growth factor (PDGF), nerve growth factor (NGF), fibroblast growth factor (FGF), insulin-like growth factor (IGF), and transforming growth factor (TGF). Examples of the differentiation factor include neurotrophin, colony stimulating factors (CSF), and transforming growth factor (TGF). Examples of cell adhesion molecule include members of the immunoglobulin (Ig) superfamily such as IgSF CAMs—the integrins, the cadherins, and the selectins.

Absorbent particle(s) (AP or APs) can absorb and retain liquid (e.g., water). When APs absorb water, they swell and increase their size. The swollen APs make pores in a device of this invention wider and the overall porosity of the device is increased when they are saturated with liquid. The size of pores and the overall porosity of the device can be also controlled by the amount of APs or the size of SAPs contained in the device.

APs can be made of or comprise, entirely or partially, such a material as polyacrylamide copolymer, ethylene maleic anhydride copolymer, cross-linked carboxymethylcellulose, polyvinyl alcohol copolymers, cross-linked polyvinypyrrolidone, cross-linked polyethylene oxide, starch grafted copolymer of polyacrylonitrile, polyurethane, Pluronic, gelatin, silica gel, cross-linked dextran (saphadex), Alginate, Agar-agar, microbial cellulose, modified clay, or their mixtures.

In some embodiments, a device of this invention is a fluffy and thick nonwoven, which comprises controllable bead structures. Such a device can contain large and open pores. In some other embodiments, such a device can have a thickness ranging from about 0.5 mm to about 20 mm; the beads can have a size of greater than 10 μm.

In some other embodiments, the three-dimensional structure in a device of this invention further comprises a substrate with at least one surface, and one or more fibrils with beads can be deposited on a surface of the substrate. The surface can be a structural support for the deposited fibrils and/or beads. For example, the substrate can be a film or culture container. In some embodiments, the substrate comprises glass, metal, or plastic (e.g., non-cytotoxic plastic).

A device of this invention can be used for in vitro cell or tissue culture, particularly cell culture that needs more cell spreading. When a cell suspension is administrated on the device, the agglomerates of cells can easily attach to the beads. The cells can penetrate and proliferate comfortably into the device. The cells can also spread randomly or orderly into the device depending on the type of the device. In some embodiments, a device of this invention comprises aligned fibers, allowing cells to penetrate along the aligned fiber.

As a specific example, a device of this invention can be used as or for a nerve regeneration device.

In some embodiments, a device of this invention or the fibrils contained therein can be fabricated according to a variety of methods know in the art, such as a spinning process, including melt spinning, electrospinning, gas jet spinning (NGJ), melt blown, or forced spinning of suitable polymers. Preferably, a device of this invention is produced by an electrospinning process.

Electrospinning and NGJ techniques permit the processing of polymers from both organic and aqueous solvents. Furthermore, based on the present invention, these techniques permit the incorporation, dispersion (both homogeneous and heterogeneous dispersions), and/or localized dispersion of discrete particles and/or soluble non-fiber forming additives into the resulting fibers via the spinning/gas jet fluid. Accordingly, the one or more beads can be incorporated in and/or on the fibrils used to form the cell culture device of the present invention.

In another aspect, the present invention provides methods for manufacturing the above-described devices of this invention by a spinning (e.g., electrospinning) method. For example, a device of this invention can be produced by the spinning of a polymer solution containing beads. Alternatively, the device can be produced by incorporating beads into the already formed (spun) fibers during the spinning process.

In some embodiments, a method for manufacturing a device of this invention may include the following steps: (1) preparing a polymer solution, which comprises beads; (2) spinning the polymer solution to form fibrils with the beads; and (3) using the fibrils with the beads to form the device for cell or tissue culture. For example, the polymer solution is a polystyrene solution a bioplastic solution such as polyhydroxyallanoate solution (PHA), or poly(lactate) (PLA) solution. As another example, the polymer solution is polycaprolactone solution, and the beads may comprise chitosan particles.

In some other embodiments, a method for manufacturing a device of this invention may include the following steps: (1) preparing a first polymer solution; (2) preparing a second polymer solution; (3) co-spinning the solution of the first polymer and the solution of the second polymer to form fibrils with beads; and (4) using the fibrils with the beads to form the device for cell or tissue culture, wherein at least the first polymer solution or the second polymer solution comprises the beads. For example, the first polymer solution is a polystyrene solution, and the second polymer solution is a polyurethane solution.

In some other embodiments, a method for manufacturing a device of this invention may include the following steps: (1) preparing a polymer solution; (2) spinning the polymer solution; (3) incorporating beads during the spinning of the polymer solution to form the fibrils with the beads; and (4) using the fibrils with the beads to form the device for cell or tissue culture. For example, the polymer solution is a polystyrene solution and the beads are absorbent particles.

Still in some other embodiments, a method for manufacturing a device of this invention may include the following steps: (1) preparing a polymer solution; (2) spinning the polymer solution; (3) incorporating beads during the spinning of the polymer solution to form the fibrils with the beads; and (4) using the fibrils with the beads to form the device for cell or tissue culture. For example, the polymer solution is a polycaprolactone solution and the beads are absorbent particles.

Further, the present invention relates to a method for culturing cells by using a device described herein. In some embodiments, the method includes a step of contacting cells (e.g., in the form of a cell suspension) with the device containing a three-dimensional structure that further comprises fibrils and beads. In some other embodiments, the method includes steps of placing the device of this invention near biological cells and allowing the cells to grow into or on the device.

As used herein, the term “bead” or “beads” can be interchanged with the term “particle” or “particles.” The beads in general refer to particles or micro-particles that may be spherical or oval or have an irregular shape. The beads can have a wide size range (e.g., a size of greater than 10 μm). The beads can be organic, inorganic, synthetic, or natural materials. For example, beads can comprise powders of natural materials (e.g., collagen, gelatin, chitosan, zein powders). The beads can also comprise one or more materials, which include but are not limited to glass beads, bioactive molecules, growth factors, differentiation factors, cell adhesion molecules or proteins, pharmaceutical small molecules and absorbent particles. In some embodiments, the beads are surface-treated or have functional groups on their surface. In some embodiments, the loading of beads in the device is in the range of 0.1%-70%. Preferably, the loading is in the range of 0.5% to 50%.

As used herein, the term “biocompatible material” is any substance that is not having any toxic or injurious effects on biological functions.

As used herein, the term “biopolymer” herein means a peptide, protein, nucleic acid or virus particle—native as well as biologically or synthetically modified—including fragments, multimers, aggregates, conjugates, fusion products, etc.

As used herein, the term “synthetic polymer” refers to polymers that are not found in nature even if the polymers are made from naturally occurring biomaterials. The term “natural polymer” refers to polymers that are naturally occurring.

As used herein, the term “glass beads” refers to particles produced by methods known in the art for making glass with a spherical or irregular shape. Glass beads may be made from any number of compositions of oxides as known in the art. Typically glass requires at least about 50% silicon oxides.

As used herein, the term “bioactive molecule” means a molecule that has an effect on a cell or tissue. Useful bioactive molecules for this invention include but are not limited to growth factors, differentiation factors, fibrous proteins, and/or adhesive proteins.

As used herein, the term “growth factor” means a molecule that promotes the proliferation of a cell or tissue. Preferably the growth factor is VEGF, collagen, bone morphogenic factor-β, EGF, PDGF, NGF, FGF, IGF, or TGF.

As used herein, the term “differentiation factor” means a molecule that promotes the differentiation of cells. Preferably the differentiation factor is neurotrophin, CSF, or TGF.

As used herein, the term “adhesive molecule or protein” or “cell adhesion molecule or protein” means a molecule or protein that promotes attachment of a cell to a bead and/or fibril. Preferably, a cell adhesion molecule is a member of the Ig (immunoglobulin) superfamily. IgSF CAMs, for example, include the integrins, the cadherins, and the selectins.

As used herein, the term “absorbent particle” means a material made from an absorbent material, which is a water-swellable, water-insoluable organic or inorganic material capable of absorbing water. For example, absorbent polymers include but are not limited to polyacrylamide copolymer, ethylene maleic anhydride copolymer, cross-linked carboxymethylcellulose, polyvinyl alcohol copolymers, cross-linked polyvinypyrrolidone, cross-linked polyethylene oxide, starch grafted copolymer of polyacrylonitrile, polyurethane, Pluronic, gelatin, silica gel, cross-linked dextran (saphadex), Alginate, Agar-agar, microbial cellulose, and modified clay.

As used herein, the term “fibrils” refers to elongated structures having a small cross section or diameter (e.g., between 50 nm to 20,000 nm or between 50 nm to 5,000 nm). Fibrils can be compatible and/or biodegradable. Further, fibrils can be surface-treated.

In one embodiment, the diameter of the fibers and/or nanofibers utilized and/or contained in the present invention ranges from about 50 nanometers to about 5,000 nanometers. In another embodiment, the fibers used in the present invention are electrospun fibers having diameters within the range of about 3 nanometers to about 5,000 nanometers, or from about 10 nanometers to about 5,000 nanometers, or even from about 50 nanometers to about 5,000 nanometers. Again, here, as well as elsewhere in the specification and claims, different individual range limits can be combined to form new ranges.

As used herein, the term “or” is meant to include both “and” and “or.”

As used herein, the term “a” or “an” can mean to include the meaning of plurality and the meaning of “any.”

### BRIEF DESCRIPTIONS OF THE FIGURES

FIG. 1 is an image of a device of this invention containing fibrils and beads.

FIG. 2 is a schematic, top view of a device of this invention produced by spinning a polymer solution containing beads.

FIG. 3 is a schematic, top view of a device of this invention produced by sprinkling beads during the spinning of a polymer solution.

FIG. 4 is a schematic, top view of a device of this invention after cell seeding. The device is produced by spinning a polymer solution which contains beads.

FIG. 5 is a schematic, cross-sectional view of a device of this invention after cell seeding. The device is produced by spinning a polymer solution which contains beads.

FIG. 6 is an image of a device of this invention. The device is produced by spinning a polycaprolactone solution which contains 1% chitosan particles.

FIG. 7 is a cross-sectional view of the device of FIG. 6.

FIG. 8 is an image of a device of this invention. The device is produced by spinning a polycaprolactone solution which contains 3% chitosan particles.

FIG. 9 is a cross-sectional view of the device of FIG. 8.

FIG. 10 is an image of a control device produced by spinning a polycaprolactone solution without chitosan particles.

FIG. 11 is a cross-sectional view of the device of FIG. 10.

FIG. 12(a) is an image of cell spreading for the device of FIG. 6.

FIG. 12(b) is an image of cell spreading for the device of FIG. 8.

FIG. 12(c) is an image of cell spreading for the device of FIG. 10.

FIG. 13 is a graph illustrating the aspect ratios of cell spreading for the devices of FIGS. 6, 8, and 10.

### DETAILED DESCRIPTION OF THE INVENTION

One aspect of the present invention relates to devices for in vitro cell or tissue culture. Each device includes a three-dimensional structure, which further includes fibrils with beads and/or particles.

As a key component of the device, the beads are capable of supporting the structure of the device so that the three-dimensional structure is open and controllable, and the device is fluffy and thick. Due to the size of the beads, cells or agglomerates of cells can be attached to the beads after cell seeding, and then penetrate and proliferate comfortably into the device (e.g., through the open, large pores within the fluffy structure) even though the device is thick. The cells can spread randomly or orderly depending on the type of the device.

In some embodiments, the beads can be active in order to enhance cell spreading, cell attachment, cell growth, and/or the differentiation of cells. For example, the beads can comprise or may be incorporated with bioactive molecules, growth factors, differentiation factors, cell adhesion molecules, pharmaceutical small molecules, or biological large molecules.

In some other embodiments, the beads can be used to support or retain the three-dimensional structure of the device when the device is saturated, and/or to open pores when the beads are swollen. For example, the beads can be absorbent particles, which are capable of absorbing and retaining liquid while increasing their size at the same time. As a result, the swollen beads make pores in the device wider and the overall porosity of the device is increased when they are saturated. The size of pores and the overall porosity of the device can be controlled by the amount or the size of absorbent particle.

Another aspect of this invention provides novel methods for manufacturing devices for cell or tissue culture. As shown in the following figures and examples, the device of this invention can be manufactured by either spinning a polymer solution containing beads, or by incorporating the beads into the fibrils during the spinning process.

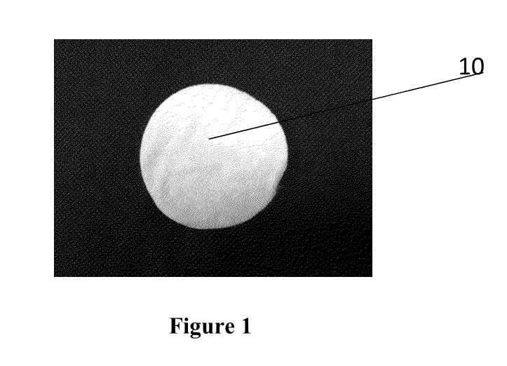

FIG. 1 shows an exemplary image for the device 10, which contains fibrils and beads. Due to the existence of the beads, the device 10 (e.g., nonwoven) is fluffy and thick, and contains large open pores within its three-dimensional structure. Embodiment of FIG. 1 shows a spherical structure, while the device can have any other types of three-dimensional structures.

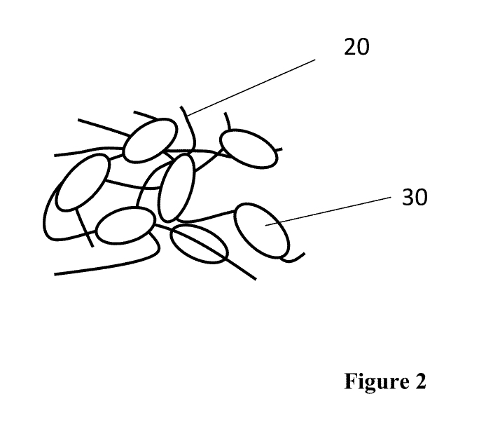

FIG. 2 illustrates a top view of a device of the invention. The device contains fibrils 20 and beads 30 and is manufactured by spinning a polymer solution containing beads 30. As shown herein, the device has large open pores, as the beads 30 are capable of supporting the fluffy three-dimensional structure. As described above, the beads can be active, or include absorbent (or its particles).

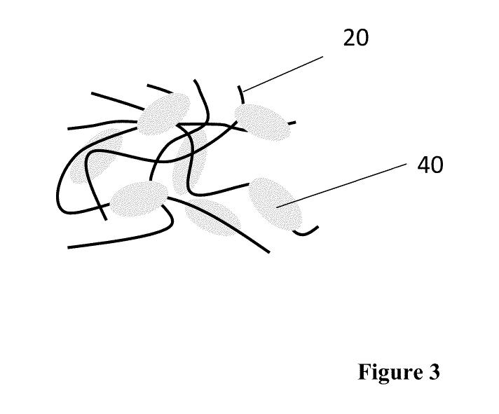

FIG. 3 illustrates a top view of another device of the invention. This device contains fibrils 20 and beads 40, where the beads 40 are incorporated during the spinning process of a polymer solution.

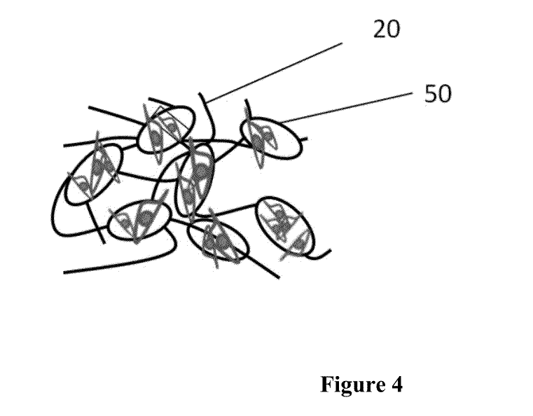

FIG. 4 illustrates a top view of a device of the invention, which contains fibrils 20 and beads 50. The device is produced by spinning a polymer solution containing the beads 50. As shown herein, after cell seeding (e.g., after a cell suspension is administrated on the device), the agglomerates of cells are attached to the beads and/or articles.

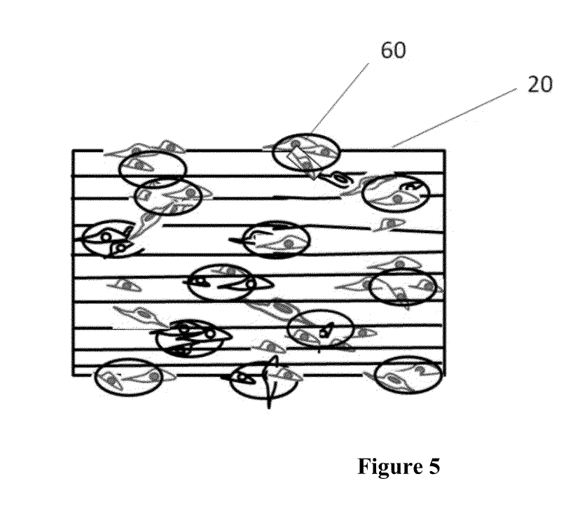

FIG. 5 illustrates a cross-sectional view of a device of the invention after seeding. The device which comprises fibrils 20 and beads 60 is manufactured by spinning a polymer solution containing the beads 60. After seeding, the cells can be attached to the beads 60 and then penetrate and proliferate comfortably into the device (e.g., through the open space or pores within the device). The cells can spread randomly or orderly, depending on the type of the device.

The present invention can further be described by way of illustration, with reference to the following example.

### Example

An absorbable polymer device of this invention is produced by spinning a polycaprolactone (PCL) solution having beads. Specifically, a 25% weight by weight PCL solution was prepared, and acetone was added to form a suspension. Chitosan particles having a size of 75 to 150 microns were prepared using US Standard Sieves. Then, chitosan particles were added to the suspension. Varying amounts of chitosan particles are used. The percentages of chitosan particles to be added are detailed below in Table 1. The resulting solutions then can be spun (e.g., electrospun) accordingly to arrive at the devices of the present invention.

TABLE 1

| Device | Percentage of Chitosan Particles to PCL Suspension |
|---|---|
| Sample A | 1% |
| Sample B | 3% |
| Sample C (control) | (0%) |

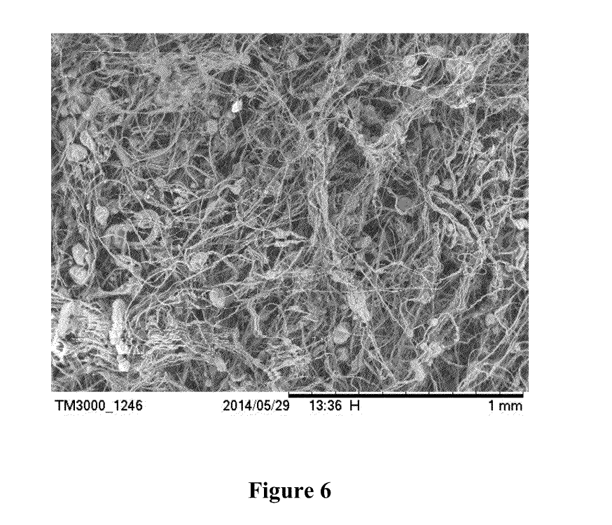

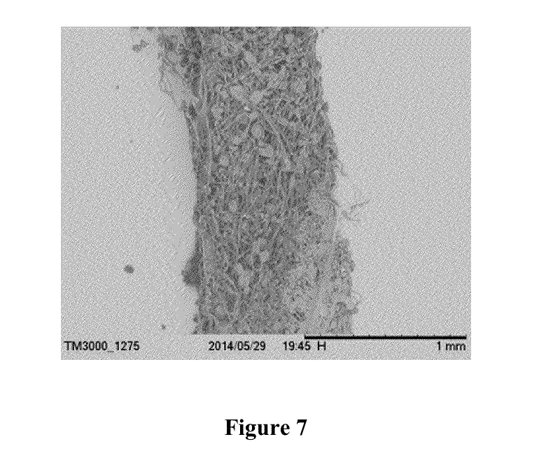

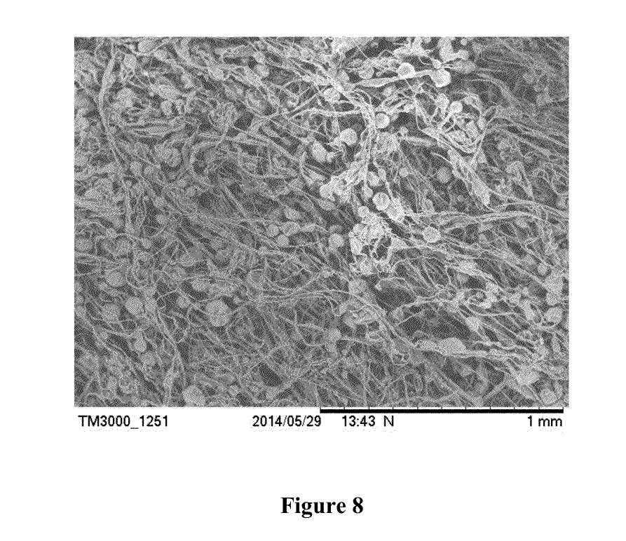

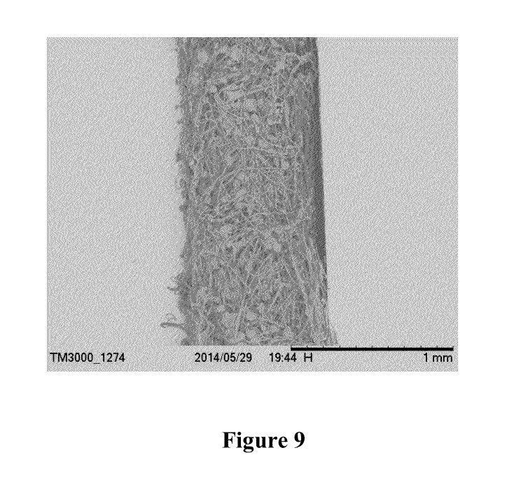

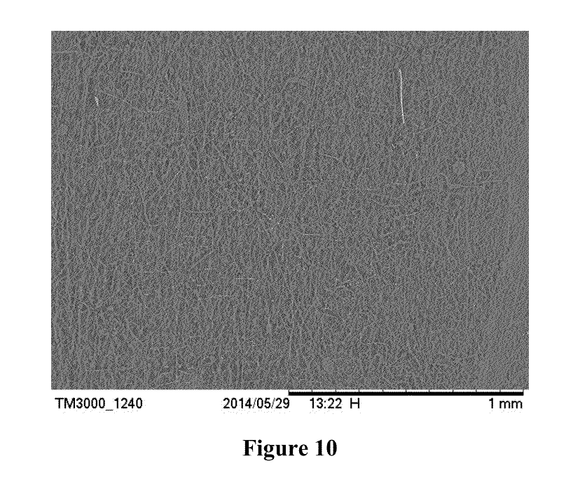

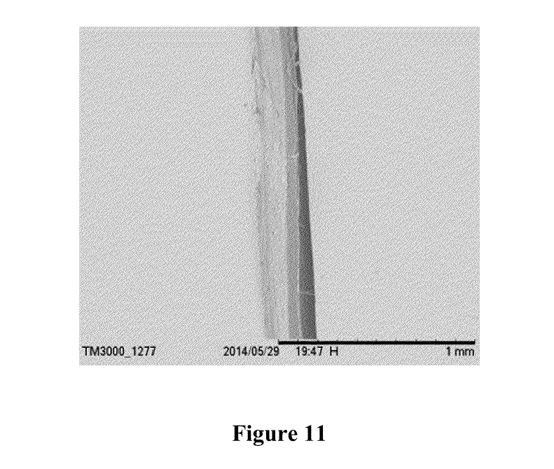

FIGS. 6 to 11 illustrate scanning electron microscope (SEM) images of the devices Sample A, B, and C. As shown in FIG. 6, Sample A contains fibrils (i.e., PCL) and 1% beads (i.e., chitosan particles). FIG. 7 shows the cross-sectional view of fibrils (i.e., PCL) and 1% beads (i.e., chitosan particles). FIG. 8 shows that Sample B contains fibrils (i.e., PCL) and 3% beads (i.e., chitosan particles). FIG. 9 shows the cross-sectional view of fibrils (i.e., PCL) and 3% beads (i.e., chitosan particles). As a control, Sample C in FIG. 10 only contains fibrils (i.e., PCL) but does not have any beads. FIG. 11 shows the cross-sectional view of the control.

### Cell Culture

Samples A, B, and C were used for cell culture. A10 smooth muscle cells (SMCs) were used, and the cell culture medium was Dulbecco's Modified Eagles Medium (DMEM, high glucose (4.5 g/L) with added 10% Fetal Bovine Serum (FBS), 1% penicillin/streptomycin (10,000 u/ml penicillin, 10,000 μg/ml streptomycin), and 1 mM sodium pyruvate. The cells were incubated at standard cell culture conditions (37° C., 5% CO2, 95% humidity), and were fed fresh media every other day. The cells were expanded in culture to obtain enough cells for seeding all samples. Samples A, B, and C were cut into small sizes (1 cm²), placed into 24 well plates, and sterilized using ethylene oxide sterilization. Those samples were then submerged in 1 ml of medium and incubated in standard cell culture conditions to allow serum proteins to attach to the materials for one day prior to cell seeding. The media was aspirated immediately before seeding. For cell seeding, the cells were trypsinized, centrifuged to obtain a pellet, and resuspended in the medium. Approximately 28,500 cells were seeded over the entire well in a 24 well plate (15,000 cells/cm²).

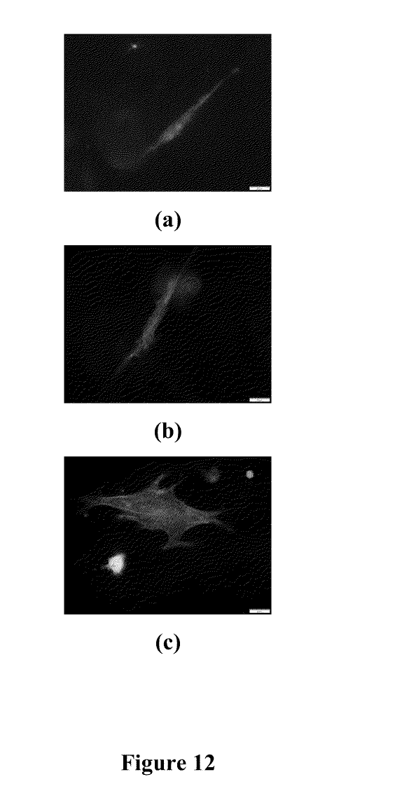

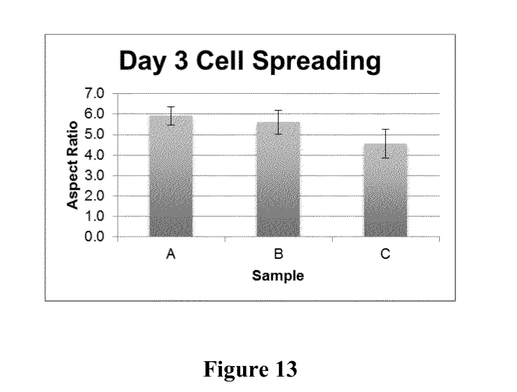

At day 3, cell spreading was calculated using the aspect ratio of the cells. A large aspect ratio indicates significant cell spreading over the sample, while a small aspect ratio indicates spreading of the cells in equal directions. To obtain the spread images, samples were IMAGED at 40× magnification using the DAPI (345/455 nm excitation/emission, cell nucleus), FITC (focal adhesions), and TRITC (cytoskeleton) channels. FIGS. 12(a), 12(b), and 12(c) provide exemplary cell spreading images for Samples A, B, and C, respectively (scale bars are 20 microns). Thirty images were acquired for each specimen and analyzed using Image J for aspect ratio, as calculated by measuring the largest length of stretch of the cell divided by the smallest distance across the cell over the nucleus. The average aspect ratio for each specimen over the thirty images was used for averaging across the different specimen for the sample average. FIG. 13 illustrates the aspect ratios of Samples A, B, and C. Among those samples, Sample A, which contains PCL fibrils and 1% chitosan particles, has the largest aspect ratio (close to 6.0). Sample B, which contains PCL fibrils and 3% chitosan particles, has an aspect ratio of about 5.5. The control (Sample C) does not contain chitosan particles and has the smallest aspect ratio (about 4.5).

As such, it is also shown that devices of the present invention with different fibrils and/or beads (e.g., Samples A and B) can result in different cellular behavior (e.g., cell spreading) for cell culture. Thus, a particular advantage of the present invention is to be used for cell culture that needs more cell spreading. For instance, a device of the present invention can be used as or for a nerve regeneration device.

Although specific embodiments of this invention have been illustrated herein, it will be appreciated by those skilled in the art that any modifications and variations can be made without departing from the spirit of the invention. The examples and illustrations above are not intended to limit the scope of this invention. Further, it is intended that this invention encompass any arrangement, which is calculated to achieve that same purpose, and all such variations and modifications as fall within the scope of the appended claim

All publications referred to above are incorporated herein by reference in their entireties. All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

1. A device for cell or tissue culture comprising a three-dimensional structure which comprises one or more fibrils and one or more beads.

2. The device of claim 1, wherein the three-dimensional structure is open and controllable.

3. The device of claim 1, wherein the three-dimensional structure comprises open pores.

4. The device of claim 1, wherein the fibrils have a size range of from 50 nm to 5,000 nm.

5. The device of claim 4, wherein at least one fibril comprises an oligomer, prepolymer, momomer, or polymeric material.

6. The device of claim 5, wherein the polymeric material is aliphatic polyester, polystyrene, polyolefin, polysaccharide, collagen, gelatin, zein, polyvinylpyrrolidone, hydroxypropyl methyl cellulose, polyethylene oxide, polyethylenimine polyvinyl alcohol, polyamides, or polyurethanes.

7. The device of claim 6, wherein the aliphatic polyester comprises polycarprolactone, poly(lactate), poly(glycolate), poly(dioxanone), polyhydroxyalkanoates, or a copolymer thereof.

8. The device of claim 1, wherein at least one bead is non-cytotoxic.

9. The device of claim 1, wherein at least one bead comprises an organic, inorganic, synthetic, or natural material.

10. The device of claim 9, wherein at least one bead comprises powder of a natural material.

11. The device of claim 10, wherein at least one bead comprises chitosan powder, collagen powder, gelatin powder, zein powder, or a combination of these.

12. The device of claim 8, wherein at least one bead comprises or is incorporated with a glass bead, bioactive molecule, growth factor, differentiation factor, cell adhesion molecule or protein, pharmaceutical small molecules, biological large molecule, or absorbent particle.

13. The device of claim 12, wherein the growth factor is VEGF, collagen, bone morphogenic factor-β, EGF, PDGF, NGF, FGF, IGF, or TGF.

14. The device of claim 12, wherein the differentiation factor is neurotrophin, CSF, or TGF.

15. The device of claim 12, wherein the cell adhesion molecule is a member of immunoglobulin superfamily.

16. The device of claim 15, wherein the immunoglobulin superfamily CAMs comprise integrins, cadherins, or selectins.

17. The devices of claim 12, where the absorbent particle comprises polyacrylamide copolymer, ethylene maleic anhydride copolymer, cross-linked carboxymethylcellulose, polyvinyl alcohol copolymers, cross-linked polyvinypyrrolidone, cross-linked polyethylene oxide, starch grafted copolymer of polyacrylonitrile, polyurethane, Pluronic, gelatin, silica gel, cross-linked dextran (saphadex), Alginate, Agar-agar, microbial cellulose, modified clay, or their mixtures.

18. The device of claim 17, wherein the absorbent particle has an increased size when absorbing the liquid.

19. The device of claim 18, wherein the absorbent particle is capable of increasing the size of open pores in the three-dimensional structure when the absorbent particle absorbs the liquid.

20. The device of claim 19, wherein the device has an increased porosity when the beads are saturated.

21. The device of claim 20, wherein at least one bead can be active in order to enhance cell spreading, cell attachments, cell growth, or the differentiation of cells.

22. The device of claim 21, wherein at least one bead is capable of increasing the size of open pores in the three-dimensional structure.

23. The device of claim 22, wherein the beads have a size greater than 10 μm.

24. The device of claim 23, wherein the beads are capable of supporting or retaining the three-dimensional structure.

25. The device of claim 24, wherein the beads have a load range of from 0.1% to 70%.

26. The device of claim 25, wherein the beads have a load range of from 0.5% to 50%.

27. The device of claim 26, wherein the fibrils are surface-treated by plasma treatment or with a biocompatible material.

28. The device of claim 27, wherein the device has a thickness range of from 0.5 mm to 20 mm.

29. The device of claim 28, wherein the cells or agglomerates of cells are attached to the beads after cell seeding.

30. The device of claim 29, wherein the device is used for a nerve regeneration device.

31. The device of claim 30, wherein the device or the fibrils are manufacture by an electrospinning, gas jet spinning, melt blown, or forced spinning process.

32. A method for manufacturing a device for cell or tissue culture of claim 1, comprising the steps of
(1) preparing a polymer solution which comprises beads;
(2) spinning the polymer solution to form fibrils with the beads; and
(3) using the fibrils with the beads to form the device for cell or tissue culture.

33. The method of claim 32, wherein the polymer solution is a polystyrene solution or a bioplastic solution.

34. The method of claim 33, wherein the bioplastic solution is a polyhydroxyallanoate solution or a poly(lactate) solution.

35. The method of claim 32, wherein the polymer solution is a polycaprolactone solution.

36. The method of claim 35, wherein the beads comprise chitosan.

37. A method for manufacturing a device for cell or tissue culture of claim 1, comprising the steps of
(1) preparing a solution of a first polymer;
(2) preparing a solution of a second polymer;
(3) co-spinning the solution of the first polymer and the solution of the second polymer to form fibrils with beads; and
(4) using the fibrils with the beads to form the device for cell or tissue culture, wherein at least the solution of the first polymer or the solution of the second polymer comprises the beads.

38. The method of claim 37, wherein the first polymer is polystyrene and the second polymer solution is polyurethane.

39. A method for manufacturing a device for cell or tissue culture of claim 1, comprising the following steps
(1) preparing a polymer solution;
(2) spinning the polymer solution to form fibrils;
(3) incorporating beads during the spinning of the polymer solution to form the fibrils with the beads,
(4) using the fibrils with the beads to form the device for cell or tissue culture.

40. The method of claim 39, wherein the polymer solution is a polystyrene solution or polycaprolactone solution.

41. The method of claim 39, wherein the beads comprise an absorbent.
